(19) United States
(12) Patent Application Publication
(10) Pub. No.: US 2002/0082620 A1
(43) Pub. Date: Jun. 27, 2002

(54) Bioactive materials for aneurysm repair

(76) Inventor: Elaine Lee, Sunnyvale, CA (US)

Correspondence Address: ROBINS & PASTERNAK LLP, 545 MIDDLEFIELD ROAD, SUITE 180, MENLO PARK, CA, 94025, US

(21) Appl. No.: 09/749,980

(22) Filed: Dec. 27, 2000

(51) Int. Cl. A61B 17/00

(52) U.S. Cl. 606/151

(57) ABSTRACT

Compositions including liquid embolics, vaso-occlusive members and combinations thereof are described. Also described are methods of using these compositions.

### FIELD OF THE INVENTION

Compositions and methods for repair of aneurysms are described. In particular, liquid embolics, vaso-occlusive members and combinations thereof are described. Also described are methods of using these materials.

### BACKGROUND

An aneurysm is a dilation of a blood vessel (similar to a balloon) that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids.

U.S. Pat. No. 5,354,295 and its parent, U.S. Pat. No. 5,122,136, both to Guglielmi et al., describe an electrolytically detachable embolic device. Modified GDC coils have also been used in aneurysms, for example surface-modified GDCs as described in Murayama et al. (1999) American J Neuradiol 20(10):1992-1999. Vaso-occlusive coils having little or no inherent secondary shape have also been described. For instance, co-owned U.S. Pat. Nos. 5,690,666 and 5,826,587 by Berenstein et al., describes coils having little or no shape after introduction into the vascular space.

A variety of mechanically detachable devices are also known. For instance, U.S. Pat. No. 5,234,437, to Sepetka, shows a method of unscrewing a helically wound coil from a pusher having interlocking surfaces. U.S. Pat. No. 5,250,071, to Palermo, shows an embolic coil assembly using interlocking clasps mounted both on the pusher and on the embolic coil. U.S. Pat. No. 5,261,916, to Engelson, shows a detachable pusher-vaso-occlusive coil assembly having an interlocking ball and keyway-type coupling. U.S. Pat. No. 5,304,195, to Twyford et al., shows a pusher-vaso-occlusive coil assembly having an affixed, proximally extending wire carrying a ball on its proximal end and a pusher having a similar end. The two ends are interlocked and disengage when expelled from the distal tip of the catheter. U.S. Pat. No. 5,312,415, to Palermo, also shows a method for discharging numerous coils from a single pusher by use of a guidewire which has a section capable of interconnecting with the interior of the helically wound coil. U.S. Pat. No. 5,350,397, to Palermo et al., shows a pusher having a throat at its distal end and a pusher through its axis. The pusher sheath will hold onto the end of an embolic coil and will then be released upon pushing the axially placed pusher wire against the member found on the proximal end of the vaso-occlusive coil.

In addition, several patents describe deployable vaso-occlusive devices that have added materials designed to increase their thrombogenicity. For example, fibered vaso-occlusive devices have been described at a variety of patents assigned to Target Therapeutics, Inc., of Fremont, Calif. Such vaso-occlusive coils having attached fibers is shown in U.S. Pat. Nos. 5,226,911 and 5,304,194, both to Chee et al. Another vaso-occlusive coil having attached fibrous materials is found in U.S. Pat. No. 5,382,259, to Phelps et al. The Phelps et al. patent describes a vaso-occlusive coil which is covered with a polymeric fibrous braid on its exterior surface. U.S. Pat. No. 5,658,308 to Snyder is directed to a coil having a bioactive core. The coils may be coated with agarose, collagen or sugar. U.S. Pat. No. 5,669,931 to Kupiecki discloses coils that may be filed or coated with thrombotic or medicinal material. U.S. Pat. No. 5,749,894 to Engleson discloses polymer coated vaso-occlusion devices. U.S. Pat. No. 5,690,671 to McGurk discloses an embolic element which may include a coating, such as collagen, on the filament surface.

U.S. Pat. No. 5,536,274 to Neuss shows a spiral implant which may assume a variety of secondary shapes. Some complex shapes can be formed by interconnecting two or more of the spiral-shaped implants. To promote blood coagulation, the implants may be coated with metal particles, silicone, PTFE, rubber latices, or polymers. U.S. Patent No. 5,980,550 describes a vaso-occlusive device having a bioactive inner coating and a water-soluble outer coating. Co-owned WO/027445, titled “Bioactive Coating for Vaso-occlusive Devices,” describes vasoocclusive devices coated with a collagen-based material and, additionally, describes the use of a tie-layer between the device and the collagen-based coating.

Liquid embolics, such as cyanoacrylate glues and fibrin sealants, have also been used in animal and human subjects. See, e.g., Interventional Radiology, Dandlinger et al, ed., Thieme, N.Y., 1990:295-313; Suga et al. (1992) No Shinkei Geka 20(8):865-873; Moringlane et al. (1987) Surg Neurol 28(5):361-366; Moringlane et al. (1988) Acta Neurochir Suppl. (Wein) 43:193-197. Of these liquid embolics, cyanoacrylate glues are the only liquid embolics currently available to neurosurgeons. However, chronic inflammation is typically seen with cyanoacrylate treatments (Herrera et al. (1999) Neurol Med Chir (Tokyo) 39(2):134-139) and the degradation product, formaldehyde, is highly toxic to the neighboring tissues. See, Vinters et al (1995) Neuroradiology 27:279-291. Another disadvantage of cyanoacrylate materials is that the polymer will adhere both to the blood vessel and to the tip of the catheter. Thus physicians must retract the catheter immediately after injection of the cyanoacrylate embolic material or risk adhesion of the cyanoacrylate and the catheter to the vessel.

Another class of liquid embolic materials—precipitative materials—was invented in late 80's. See, Sugawara et al (1993) Neuro Med Chir (Tokyo) 33:71-76; Taki et al (1990) AJNR 11:163-168; Mandai et al (1992) J Neurosurgery 77:497-500. Unlike cyanoacrylate glues which are monomeric and rapidly polymerize upon contact with blood, precipitative materials are pre-polymerized chains that precipitate into an aggregate upon contact with blood. One potential problem in using the precipitating polymers is the use of organic solvents to dissolve the polymers, i.e., ethanol for PVAc and DMSO for EVAL and CA. These materials are strong organic solvents that can dissolve the catheter hub, and, in the case of DMSO, can damage microcapillary vessels and surrounding tissues. These solvents are also known to cause vasospasm of blood vessels. Additionally, these precipitating agents are often difficult to deliver and typically require the use of multi-lumen catheters (see, e.g., U.S. Pat. No. 6,146,373).

Thus, there remains a need for methods and compositions useful in promoting rapid ingrowth of dense connective tissue in aneurysms.

### SUMMARY OF THE INVENTION

Thus, this invention includes novel occlusive compositions as well as methods of using and making these compositions.

In one aspect, the invention includes a vaso-occlusive composition comprising a vaso-occlusive member and an additional material comprising, for example, fibrin; polyethylene glycol derivatives; thrombin-coated gelatin granules; balloons coated with iron microspheres, trace metals, thrombus-stabilizing molecules and combinations of two or more of these materials. The vaso-occlusive member can be any implantable devices, for example, a vaso-occlusive coil, a stent, a filter or the like. In certain embodiments, the additional material is a trace metal such as copper. In other embodiments, the additional material is a thrombus-stabilizing molecule or functional fragments thereof, for example Factor XIII, α₂-antiplasmin or plasminogen activator inhibitor-1 (PAI- 1). Furthermore, the additional material can be adsorbed or otherwise attached to the vaso-occlusive member.

In certain aspects, the composition also includes a further bioactive material, for example, one or more cytokines (e.g., PDGF, bFGF, VEGF, TGF-beta, or functional fragments thereof); extracellular matrix material (e.g., collagen); genetic material (e.g., DNA and/or RNA); combinations of two or more bioactive materials; functional fragments of one or more bioactive materials, etc. In certain embodiments, the bioactive material is a cytokine such as PDGF, bFGF, VEGF, TGF-beta. The bioactive material can be adsorbed or otherwise attached to the vaso-occlusive member and, in certain embodiments, both the material and bioactive material are adsorbed or otherwise attached to the vaso-occlusive member.

In other aspects, any of the compositions described herein can include a vaso-occlusive member that has additional surface-modifications, for example by plasma treatment, ion implantation, microtexturing, use of tie layer, etc. In any of the compositions described herein, the vaso-occlusive member can be, for example, one or more vaso-occlusive coils, one or more filters, one or more retention devices and combinations thereof.

In another aspect, the invention includes a method of occluding a vessel comprising administering to a subject in need thereof any of the vaso-occlusive compositions described herein. In certain embodiments, the vessel is an aneurysm, for example a small-diameter neurovascular aneurysm.

In yet another aspect, the invention includes a method of occluding an aneurysm comprising administering to a subject in need thereof fibrin; polyethylene glycol derivatives; thrombin-coated gelatin granules; balloon coated with iron microspheres; trace metals (e.g., copper); thrombus-stabilizing molecules (e.g.,Factor XIII; α₂-antiplasmin, PAI-1 or combinations thereof). In certain embodiments, the method further includes administering a bioactive material, for example, at least one cytokine (e.g., PDGF, bFGF, VEGF and TGF-beta, combinations of two or more cytokines or functional fragments of one or more cytokines); extracellular matrix molecules (e.g., collagen); genetic material (e.g., DNA and/or RNA); combinations of two or more bioactive materials; and functional fragments of two or more bioactive materials.

These and other embodiments of the subject invention will readily occur to those of skill in the art in light of the disclosure herein.

### DESCRIPTION OF THE INVENTION

Occlusive (e.g., embolic) compositions are described. The embolic compositions include, for example, one or more liquid embolics and/or one or more vaso-occlusive devices. Additional bioactive materials may also be used in these embolic compositions. The compositions described herein find use in vascular and neurovascular indications and are particularly useful in treating aneurysms, for example small-diameter, curved or otherwise difficult to access vasculature, for example cerebral aneurysms. Methods of making and using these devices also an aspects of this invention.

Advantages of the present invention include, but are not limited to, (i) promoting healing of aneurysms using surface-modified vaso-occlusive devices; (ii) treating aneurysms with liquid embolics and/or vaso-occlusive devices; and (iii) improving treatment of aneurysms using additional bioactive materials in combination with liquid embolics and/or vaso-occlusive devices.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to “a coil” includes a mixture of two or more such devices and the like.

In one aspect, the invention includes liquid embolic materials useful in occluding aneurysms. The term “liquid embolic” refers to any agent capable of acting as an occlusive agent which is in fluid form at some point during delivery and which fully or partially solidifies. Thus, the term includes particulate materials (e.g., granules, beads, microspheres, etc.) that can be administered in an aqueous solution or in suspension. Liquid adhesives and sealants (e.g., embolics) have been approved for use to control bleeding during surgery. However, described herein is the use of these and other liquid embolics for occluding aneurysms, for example aneurysms located in tortuous pathways or small-diameter aneurysms. In certain embodiments, the liquid embolic comprises fibrin. Fibrin-containing compositions are commercially available, for example from Baxter. Collagen containing compositions are commercially available, for example from Cohesion Technologies, Inc., Palo Alto, Calif. In other embodiments, the liquid embolic comprises one or more polyethylene glycol (PEG) derivatives, for example PEG derivatives available from Cohesion Technologies, Inc., Palo Alto, Calif. Thrombin-containing materials (e.g., thrombin coated gelatin granules, available for example from Fusion) and iron-containing materials (e.g., balloons coated with iron microspheres), also find use in the present invention. These liquid embolic materials can be used alone or in any combination.

Furthermore, the liquid embolic materials can also be used in combination with additional bioactive materials. The term “bioactive” refers to any agent which exhibits effects in vivo, for example a thrombotic agent, a therapeutic agent or the like. Non-limiting examples of bioactive materials include cytokines; extracellular matrix molecules (e.g.,collagen); trace metals (e.g., copper); and other molecules that stabilize thrombus formation or inhibit clot lysis (e.g., proteins or functional fragments of proteins, including but not limited to Factor XIII, α₂-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (PDGF), vascular endothelial growth factor (VEGF), transforming growth factor beta (TGF-β) and the like. Cytokines, extracellular matrix molecules and thrombus stabilizing molecules (e.g., Factor XIII, PAI-1, etc.) are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequence of many of these molecules are also available, for example, from the GenBank database. Thus, it is intended that the invention include use of DNA or RNA encoding any of the bioactive molecules. Furthermore, it is intended, although not always explicitly stated, that molecules having similar biological activity as wild-type or purified cytokines, extracellular matrix molecules and thrombus-stabilizing proteins (e.g., recombinantly produced or mutants thereof) and nucleic acid encoding these molecules are intended to be used within the spirit and scope of the invention. Further, the amount and concentration of liquid embolic and/or other bioactive materials useful in the practice of the invention can be readily determined by a skilled operator and it will be understood that any combination of materials, concentration or dosage can be used, so long as it is not harmful to the subject.

In other embodiments, the liquid embolics and/or other bioactive materials are used in combination with one or more vaso-occlusive devices, for example vaso-occlusive coils, stents, filters, balloons, retention devices and/or aneurysm liners. Thus, for example, any of the space-filling liquid embolic materials and/or any of the other bioactive materials described herein can be used in combination with devices such as vaso-occlusive coils. The device(s) and space-filling material(s) can be administered concurrently or one after the other.

Surface-modified vaso-occlusive devices also form an aspect of this invention, either alone or in combination with one or more liquid embolics and/or one or more other bioactive materials. As used herein, the term “surface-modified” refers to any modification to a vaso-occlusive member including, but not limited to, adsorbing (e.g., coating) one or more materials on the surface of such a member; use of a tie layer for bonding of the liquid embolic and/or bioactive material to the vaso-occlusive member; ion implantation treatment; plasma or corona treatment; micropatterning or microtexturing (e.g., sandblasting) or the like. Further, methods of adsorbing (e.g., coating) the liquid embolic(s) and/or bioactive material(s) onto the vaso-occlusive device include, but are not limited to, dip coating, extrusion coating, spray deposition and the like.

Plasma treatment of coils is described for example in co-pending U.S. Ser. No. 08/598,325. These plasma-treated coils exhibit an amino-functionality which may be measured using known chemical methods. When the devices treated by this process are placed in a bloodstream, the amino-functionality results in a slight positive ionic charge on the surface of the fibers. This amino-functionality attracts platelets and thrombogenic proteins from the bloodstream. Plasma treatment may be carried out using, for example, a plasma generator such as that found in U.S. Pat. No. 3,847,652. The plasma may comprise a nitrogen containing gas, preferably those containing diatomic nitrogen or ammonia. Gas pressures are advantageously maintained at a very low level, for example, no greater than about 5 millimeters of mercury, preferably from 0.1 to 2 millimeters of mercury. The period of time in which the vaso-occlusive device is subjected to the plasma need not be great. That is to say that for most applied power settings below about 200 watts and in the radio frequency region between 1 and 50 megahertz, the time of reaction need not be greater than 10 minutes to achieve the result described herein.

Ion implantation is a physicochemical surface modification process resulting from the impingement of a high-energy ion beam and is described, for example, in Murayama et al. (1997) Neurosurgery 40(6):1233-1243. Similarly, methods of microtexturing or micropatterning, for example using X-ray, electron-beam and photo-lithography, microcontact printing, embossing, micromouldings, cold welding or sandblasting techniques, are also known to those of skill in the art. It will be apparent that any combination of the surface modifications described herein can be used. Thus, a vaso-occlusive member can be include one or more physical surface modifications (e.g., plasma treatment, ion implantation, etc.) in addition to having one or more liquid embolics and/or bioactive materials adsorbed thereto. In preferred embodiments, the vaso-occlusive device is coated with a liquid embolic and/or one or more additional bioactive materials.

The vaso-occlusive members useful in the present invention may be made using conventional equipment and procedures. For example, helical coils may be prepared by wrapping a suitable wire about a cylindrical or conical mandrel. The core of the coil (e.g., helix) may be empty or filled with one or more materials, for example fiber strands. The strand(s) are then placed axially through the core of the helix and, if a multiplicity of strands are employed, their ends bound by heat, adhesives, or mechanical means. Radial filaments may also be attached to the windings of the helix by tying or with adhesives.

The devices are those materials which are generally approved for use as implants in the body or could be so approved. They may be of polymers such as polyethylene, polyacrylics, polypropylene, polyvinylchloride, polyamides such as Nylon, polyurethanes, polyvinylpyrrolidone, polyvinyl alcohols, polyvinylacetate, cellulose acetate, polystyrene, polytetrafluoroethylene, polyesters such as polyethylene terephthalate (Dacron), silk, cotton, and the like. When the polymers are fibrous, they are often looped or tufted. Although it is not critical to this invention, they are usually assembled in bundles of 5 to 100 fibers per bundle. Preferred materials for the polymer component of vaso-occlusive devices comprise polyesters, polyethers, polyamides, and polyfluorocarbons. Especially preferred is polyethyleneterephthalate, sold as Dacron.

The coils may be made of any of a wide variety of biocompatible metals. In particular, the metals may be selected from gold, rhenium, platinum, palladium, rhodium, ruthenium, various stainless steels, tungsten, and alloys thereof. The preferred alloy is one comprising upwards of 90 percent platinum and at least a portion of the remainder tungsten. This alloy exhibits excellent biocompatibility and yet has sufficient strength and ductility to be wound into coils of primary and secondary shape and will retain those shapes upon placement of the vaso-occlusive device in the human body. The diameter of the wire typically making up the coils is often in a range of 0.005 and 0.050 inches. The resulting primary coil diameter typically is in the range of 0.008 and 0.085 inches. Smaller coil diameters are used for finer problems and larger coil diameters and wire diameters are used in larger openings in the human body. A typical coil primary diameter is 0.015 and 0.018 inches. The axial length of a vaso-occlusive device may be between 0.5 and 100 centimeters. The coils are typically wound to have between 10 and 75 turns 20 per centimeter.

In addition, the vaso-occlusive device may comprise a substrate comprising a woven braid rather than the helical coil. The vaso-occlusive device may comprise a mixture of coil and braid. Indeed, it is within the scope of this invention that all or a portion of the coil be polymeric, or a combination of metal and polymer. Additionally, the vaso-occlusive device may be made of a absorbable material, for example as described in WO 99/44538.

It is further within the scope of this invention that the vaso-occlusive device comprise shapes or structures other than coils or braids, for examples, solid sphere structures and the like.

The embolic compositions (e.g., liquid embolics, vaso-occlusive members and/or other bioactive materials) described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place implantable device at the target site but yet with a sufficient portion of the distal end of the delivery mechanism protruding from the distal end of the catheter to enable detachment of the implantable device. For use in peripheral or neural surgeries, the delivery mechanism will normally about 100-200 cm in length, more normally 130-180 cm in length. The diameter of the delivery mechanism is usually in the range of 0.25 to about 0.90 mm. Briefly, the liquid embolics and/or occlusive devices described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g., mechanical devices and/or liquid embolics and bioactive materials) which cause formation of an emboli and, at some later time, is at least partially replaced by neovascularized collagenous material formed around the implanted devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the liquid embolic and/or implantable device at the distal end, is advanced through the catheter. The embolic(s) is(are) advanced past the distal end of the catheter and positioned or extruded precisely at the desired treatment site. They are held in place by gravity, shape, size, volume, magnetic field or combinations thereof. As noted above, the order in which the components of the vaso-occlusive composition (e.g. liquid embolic; vaso-occlusive member; retention device; and/or other bioactive materials) are released from the catheter is not critical to the practice of the invention and can be determined by the operator.

Modifications of the procedure and device described above, and the methods of using them in keeping with this invention will be apparent to those having skill in this mechanical and surgical art. These variations are intended to be within the scope of the claims that follow.

What is claimed is:

1. A vaso-occlusive composition comprising a vaso-occlusive member and a material selected from the group consisting of fibrin; polyethylene glycol derivatives; thrombin-coated gelatin granules; balloons coated with iron microspheres, trace metals, thrombus-stabilizing molecules and combinations thereof.

2. The composition of claim 1, further comprising a bioactive material selected from the group consisting of
(i) at least one cytokine;
(ii) extracellular matrix material;
(iii) DNA;
(iv) RNA;
(iv) combinations of (i), (ii) and (iii); and
(v) functional fragments (i), (ii) (iii) and (iv).

3. The composition of claim 2, wherein the bioactive material is at least one cytokine.

4. The composition of claim 3, wherein the cytokine is selected from the group consisting of PDGF, bFGF, VEGF and TGF-beta.

5. The composition of claim 1, wherein the material comprises a trace metal.

6. The composition of claim 5, wherein the trace metal comprises copper.

7. The composition of claim 1, wherein the material comprises a thrombus-stabilizing molecule.

8. The composition of claim 7, wherein the thrombus-stabilizing molecule is Factor XIII or functional fragments thereof.

9. The composition of claim 7, wherein the thrombus-stabilizing molecule is plasminogen activator inhibitor-1 (PAI-1) or functional fragments thereof.

10. The composition of claim 7, wherein the thrombus-stabilizing molecule is α₂-antiplasmin or functional fragments thereof.

11. The composition of claim 1, wherein the material is adsorbed to the vaso-occlusive member.

12. The composition of claim 2, wherein the bioactive material is adsorbed to the vaso-occlusive member.

13. The composition of claim 2, wherein the material and the bioactive material are adsorbed to the vaso-occlusive member.

14. The composition of claim 1, wherein the vaso-occlusive member is plasma treated.

15. The composition of claim 1, wherein the vaso-occlusive member is subjected to ion implantation.

16. The composition of claim 1, wherein the vaso-occlusive member is microtextured.

17. The composition of claim 11, wherein the vaso-occlusive member further comprises a tie-layer between the vaso-occlusive member and the material.

18. The composition of claim 1, wherein the vaso-occlusive member is selected from the group consisting of one or more vaso-occlusive coils, one or more filters, one or more retention devices and combinations thereof.

19. A method of occluding a vessel comprising administering to a subject in need thereof a vaso-occlusive composition according to claim 1.

20. The method of claim 19, further comprising administering a bioactive material selected from the group consisting of
(i) cytokines;
(ii) extracellular matrix molecules;
(iii) DNA;
(iv) RNA;
(v) combinations of (i), (ii), (iii) and (iv);
(vi) and functional fragments of (i), (ii), (iii), (iv) and (v).

21. The method of claim 19, wherein the cytokine is selected from the group consisting of PDGF, bFGF, VEGF and TGF-beta.

22. The method of claim 19, wherein the trace metal is copper.

23. The method of claim 19, wherein the thrombus-stabilizing molecule is selected from the group consisting of Factor XIII, α₂-antiplasmin, plasminogen activator inhibitor-1 (PAI-1), combinations thereof and functional fragments thereof.

24. The method of claim 19, wherein the vessel is an aneurysm.

25. A method of occluding an aneurysm comprising administering to a subject in need thereof a material selected from the group consisting of fibrin; polyethylene glycol derivatives; thrombin-coated gelatin granules; balloon coated with iron microspheres; trace metals; thrombus-stabilizing molecules; and combinations thereof.

26. The method of claim 25, further comprising administering a bioactive material selected from the group consisting of
(i) cytokines;
(ii) extracellular matrix molecules,
(iii) DNA;
(iv) RNA;
(v) combinations (i), (ii), (iii) and (iv); and
(vi) functional fragments of (i), (ii), (iii), (iv) and (v).

27. The method of claim 26, wherein the cytokine is selected from the group consisting of PDGF, bFGF, VEGF and TGF-beta.

28. The method of claim 25, wherein the trace metal is copper.

29. The method of claim 25, wherein the thrombus-stabilizing molecule is selected from the group consisting of
(i) Factor XIII;
(ii) α₂-antiplasmin;
(iii) combinations of (i) and (ii); and
(iv) functional fragments of (i), (ii) and (iii).

30. The method of claim 25, wherein the aneurysm is a neurovascular aneurysm.
